(19) United States
(12) Patent Application Publication
(10) Pub. No.: US 2012/0150025 A1
(43) Pub. Date: Jun. 14, 2012

(54) Image Registration Using Interventional Devices

(75) Inventors: Klaus J. Kirchberg, Plainsboro, NJ (US); Rui Liao, Princeton Junction, NJ (US)

(73) Assignee: Siemens Corporation, Iselin, NJ (US)

(21) Appl. No.: 13/236,761

(22) Filed: Sep. 20, 2011

Related U.S. Application Data
(60) Provisional application No. 61/384,849, filed on Sep. 21, 2010.

(51) Int. Cl. A61B 5/05

(52) U.S. Cl. 600/424

(57) ABSTRACT

A system receives an image volume of a patient. A catheter applied to the patient contains at least one sensor, which may be a microcoil and which is detectable in the image volume. A size and a shape of a region of interest are pre-defined. A processor determines a location of the at least one sensor in the image volume. The image volume is generated by a medical imaging device. The processor defines the shape and size of the region of interest relative to the location of the at least one sensor to determine the region of interest in the image volume. Image data of the region of interest in the image volume and of the region of interest in a previous image volume are registered. The region of interest is determined during an interventional procedure on the patient.

### STATEMENT OF RELATED CASES

This case claims priority to and the benefit of U.S. Provisional Patent Application Ser. No. 61/384,849, filed Sep. 21, 2010, which is incorporated herein by reference.

### TECHNICAL FIELD

The present invention relates to systems and methods for medical imaging and more specifically to automatic determination of a region of interest (ROI) in a medical image.

### BACKGROUND

Image registration aims at aligning one or more objects which are present in different images which contain these objects. Registration techniques can be useful in medical procedures in which a pre-operative image space needs to be properly correlated to a real-time physical space for instance during a medical intervention. In image-guided surgical procedures, for instance in MM guided procedures, pre-operatively acquired images may have to be registered onto intra-operative, near real-time images. In this way, a surgeon can be guided during his operation by viewing, in real time, images of the anatomical region being treated and/or the surgical devices.

Definition or specification of a region of interest (ROI) during an intervention by a user interaction can be troublesome, inconvenient or even prohibitive during an actual intervention. Existing methods to automatically determine a region of interest require certain image features to be present in the ROI which is usually not applicable in an interventional scenario.

Accordingly, improved and novel methods and systems for automatically determining a region of interest (ROI) are required.

### SUMMARY OF THE INVENTION

A trackable interventional device, e.g. a catheter with embedded microcoil under MR, or any active sensor that is embedded in the catheter, can be used to facilitate and enhance registration between intra-operative and pre-operative images during interventions in several ways.

First, its position can be used to define the region of interest (ROI) for registration. This facilitates an accurate and robust registration right at the target, by discarding less-relevant peripheral structures, and/or mis-matched image contents due to field-of-view (FOV) difference.

Second, its position is used for initial coarse alignment between the intra-operative and pre-operative images by utilizing the prior knowledge of its physical position relative to the preoperative data.

Third, when there is change in the reference coordinate system during the interventional procedure, its position is used to estimate the change of the reference coordinate system for more accurate re-registration.

In accordance with an aspect of the present invention a method is provided for determining a region of interest in an image volume of a patient, comprising defining on a processor a size and a shape of the region of interest, applying to the patient a catheter with at least one sensor which is enabled to be detected in the image volume, the processor receiving data representing the image volume, the processor determining a location of the at least one sensor in the image volume, the processor determining the region of interest in the image volume based on the location of the at least one sensor in the image volume, the processor receiving image data of a previously acquired image volume of the patient and the processor registering a part of the previous acquired image volume defined by the region of interest with the region of interest in the image volume.

In accordance with a further aspect of the present invention the method is provided, wherein the at least one sensor is a microcoil.

In accordance with yet a further aspect of the present invention the method is provided, wherein the at least one sensor is a passive sensor.

In accordance with yet a further aspect of the present invention the method is provided, wherein the catheter and the at least one sensor is moved relative to the patient to obtain a second location in the image volume and the processor determines a second region of interest.

In accordance with yet a further aspect of the present invention the method is provided, wherein the shape of the region of interest depends upon a bounding shape of a location of the catheter in the patient.

In accordance with yet a further aspect of the present invention the method is provided, wherein the shape of the region of interest depends upon a geometry of an imaged organ.

In accordance with yet a further aspect of the present invention the method is provided, wherein the processor processes image data inside the region of interest.

In accordance with yet a further aspect of the present invention the method is provided, wherein the location of the at least one sensor is used for an alignment of the previously acquired image volume with an interventional device.

In accordance with yet a further aspect of the present invention the method is provided, wherein the location of the at least one sensor in the image volume is used by the processor as a reference frame relative to the patient.

In accordance with yet a further aspect of the present invention the method is provided, wherein the processor determines the region of interest during an interventional procedure on the patient.

In accordance with an aspect of the present invention a system to determine a region of interest in an image volume of a patient is provided, comprising: a memory enabled to store and retrieve data, a processor enabled to execute instructions to perform the steps: defining a size and a shape of the region of interest, receiving data representing the image volume, determining a location of at least one sensor of a catheter in the image volume, determining the region of interest in the image volume based on the location of the at least one sensor in the image volume and registering a part of a previously acquired image volume defined by the region of interest with the region of interest in the image volume.

In accordance with another aspect of the present invention the system is provided, wherein the at least one sensor is a microcoil.

In accordance with yet another aspect of the present invention the system is provided, wherein the at least one sensor is a passive sensor.

In accordance with yet another aspect of the present invention the system is provided, wherein the at least one sensor is moved relative to the patient to obtain a second location in the image volume and the processor determines a second region of interest.

In accordance with yet another aspect of the present invention the system is provided, wherein the shape of the region of interest depends upon a bounding shape of a location of a catheter in the patient.

In accordance with yet another aspect of the present invention the system is provided, wherein the shape of the region of interest depends upon a geometry of an imaged organ.

In accordance with yet another aspect of the present invention the system is provided, wherein the processor processes image data inside the region of interest.

In accordance with yet another aspect of the present invention the system is provided, wherein the location of the at least one sensor is used for an alignment of the previously acquired image volume with an interventional device.

In accordance with yet another aspect of the present invention the system is provided, wherein the location of the at least one sensor in the image volume is used by the processor as a reference frame relative to the patient.

In accordance with yet another aspect of the present invention the system is provided, wherein the processor determines the region of interest during an interventional procedure on the patient.

### DESCRIPTION OF THE DRAWINGS

FIG. 1 illustrates a catheter with multiple sensors in accordance with an aspect of the present invention;

FIG. 2 illustrates an image volume in accordance with an aspect of the present invention;

FIG. 3 illustrates steps in accordance with an aspect of the present invention;

FIG. 4 illustrates a user interface provided by a processor that enables the selection of a desired ROI; and

FIGS. 5 and 6 each illustrate a system in accordance with an aspect of the present invention.

### DESCRIPTION

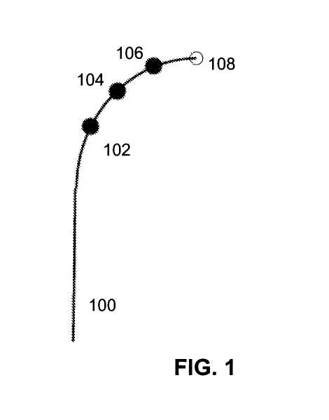

In accordance with an aspect of the present invention, the location of the ROI in an image such as an MRI image can be determined from the location of a sensor (e.g. microcoil) or any derived location from one or multiple sensors, e.g. using the knowledge about the shape of the device. This can be achieved by embedding micro-coils into the catheter and then measuring the positions of these coils (and thus the catheter) with one dimensional (1D) projections along all three spatial directions (X, Y and Z). This is illustrated in FIG. 1 with catheter 100, with coils 102, 104 and 106 and catheter tip 108. Microcoils are well known. For example, Surgi-Vision, Inc has a line of miniaturized microcoils, also referred to as magnetic resonance probes, that can produce images from inside the body. These coils can be used with any 1.5 Tesla MR equipment.

Accurate localization of an active device such as a microcoil is described in the article “Accurate Localization of Active Devices during Interventional MR Imaging” by Barbot et al. in Proc. Intl. Soc. Mag. Reson. Med. 19 (2011), page 1747, which is incorporated herein by reference.

In one embodiment of the present invention an active sensor such as a microcoil is used in a catheter. The activated coil shows up in an MR image and known techniques can be used to identify the illuminated sensor.

In another embodiment of the present invention, passive elements or sensors can be applied. For instance a tube filled with Gadolinium is detectable in an MR image and can be used to determine a location of the catheter through image segmentation techniques. Thus, the passive sensor will be visible in the obtained image and any one of a variety of segmentation techniques can be used to identify or detect the passive sensor in the image.

In accordance with an aspect of the present invention, the ROI changes when the device is moved. This allows a surgeon or a medical practitioner to follow a moving catheter during an interventional procedure and having pre-operative images being registered to the moving ROI.

In accordance with an aspect of the present invention, a ROI is determined with the shape of the ROI being fixed. For example, the shape can be a box (cubic or non-cubic shaped) or a sphere (or any other shaped having curvilinear shape) of certain dimensions centered at, for instance, at the device tip or any other defined part of the catheter in relation to the sensors. In an embodiment of the present invention the ROI falls within a box with a maximum side length of between 2-10 cm. In an embodiment of the present invention the ROI falls within a box with a maximum side length of between 2-5 cm. In an embodiment of the present invention the ROI falls within a box with a maximum side length of greater than 10 cm or less than 2 cm. The dimensions of the ROI can be provided as a distance or as a number of pixels. In accordance with an aspect of the present invention, the shape is defined by the history of device locations, e.g. the bounding box of the current and all prior device locations.

In other embodiments of the present invention, the shape can be selected by a user. Once again, the shape can be any type of volume. Thus, a box (cubic or non cubic) can be selected as a desired shape of the ROI. Alternatively, a sphere shape or any other curvilinear shape can be selected. As before, the selected shape of the ROI depends on the procedure being performed.

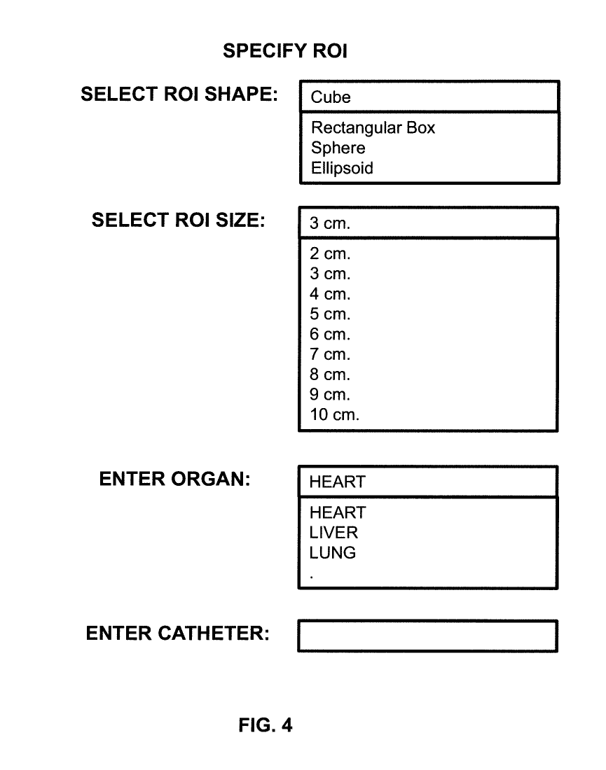

An example of a user interface that a processor presents on a display in accordance with an aspect of the invention to allow a user to select the shape of the ROI is illustrated in FIG. 4.

Thus, the selected size and the shape of the ROI depends on the procedure being performed. Thus, the size of the selectable ROI can be varied as needed and as desired. In accordance with an aspect of the present invention, a processor provides a user with an interface that allows the user to select the size and shape of the ROI. In accordance with one aspect of the present invention, the processor presents a drop down list box with various shapes that can be selected. The shapes include a cubic box, a rectangular box, a sphere and other non-linear shapes. The processor can also present one or more drop down list boxes to allow the size of the shape of the selected ROI to be selected by a user. The drop down list box can include a plurality of selectable sizes as guidance for the user. Thus, if the ROI is the shape of a cube is being selected, only one dimension need be selected. As mentioned earlier, the dimension can be specified in absolute distances, in pixels or by any other measurement. If the ROI shape is more complicated, additional user interfaces can be provided to allow the shape to be sized.

An example of a user interface in accordance with an aspect of the present invention, that allows selection of a size of the ROI, is illustrated in FIG. 4.

In accordance with an aspect of the present invention, additional structural information is incorporated, e.g. if the geometry of certain organs is known, the ROI can be defined by the organ that the device is currently in. This provides further context to the surgeon.

Thus, a processor provides a user interface to the surgeon or other user that allows the user to specify the organ of interest. The processor can then automatically select a shape and a size of the ROI according to the organ of interest in accordance with an aspect of the present invention. Alternatively, the processor can check the selected ROI shape and size to ensure it is appropriate. This user interface is shown in FIG. 4.

In accordance with an aspect of the present invention, the ROI as determined in accordance with an aspect of the present invention is used for operations other than registration, e.g. local image enhancement, adjusting acquisition parameters, location-specific visualization. These operations and parameters thereof can be made dependent upon the type of procedure or upon the organ on general location that is subject to the intervention.

In accordance with an aspect of the present invention, a prior knowledge about the physical position of the device relative to the pre-operative images to be registered is used for initial rough alignment. For example: during an atrial fibrillation (AFib) ablation procedure, an ablation catheter is typically located in the left atrium while a coronary sinus (CS) catheter is typically inserted into the coronary sinus. When the anatomy about the pre-operative images is known, e.g. via segmentation, initial alignment can be achieved by aligning the anatomy in the pre-op images with the interventional device. Accordingly, the knowledge of the CS catheter sitting in the coronary sinus can be used for initial alignment.

When there is change in the reference coordinate system during interventions, e.g. due to repositioning of the patient in a MR-guide procedure, assuming the device is not noticeably moved physically right before and after the re-referencing, then the change of the position of the devices closely represents the change in the reference coordinate system. This information can be further used by the registration algorithm for more accurate initial alignment and registration.

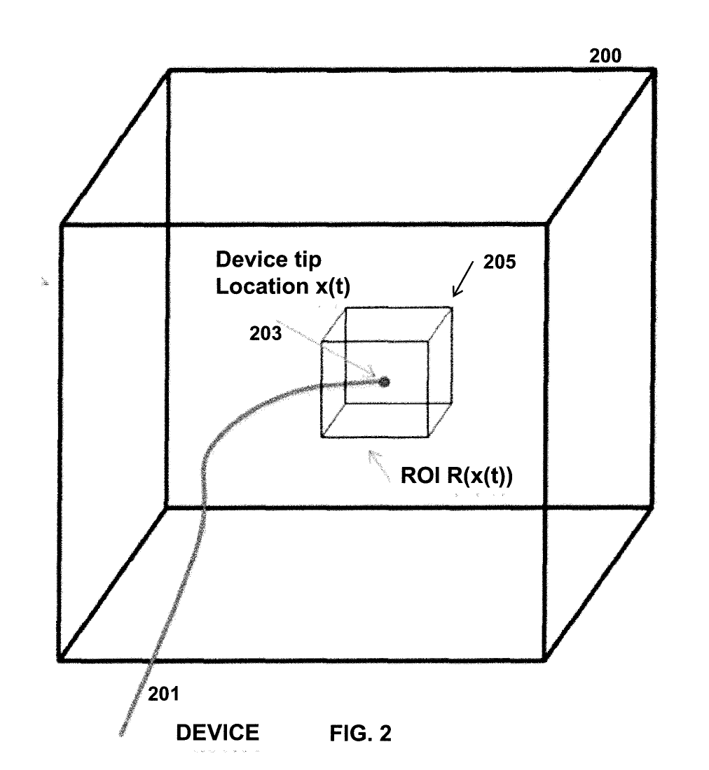

The determination of a region of interest is further illustrated in FIG. 2. A 3D image or volume 200 of a patient which includes a catheter or device 201 with a device tip 203 which may contain one or more sensors, such as a microcoil, which enables a location of the device tip 203 in the volume 200. A processor determines the location of the device tip 203 in the volume 200. Based on predefined criteria (such as which procedure is being performed, or the organ in which the device tip is located) a size and shape of an ROI 205 is determined. The determined and established ROI is then applied for image registration within the ROI, or for other processes that use the region of interest. For instance the ROI can be associated with a search range to perform an automatic image registration with a related pre-operative image.

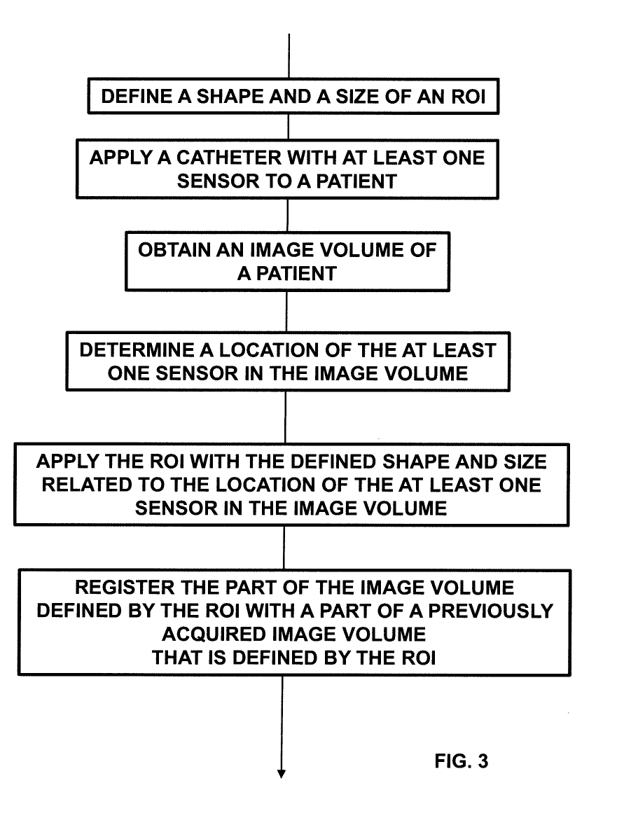

FIG. 3 illustrates the steps of applying a region of interest for image registration in accordance with an aspect of the present invention. Herein, part of the image volume with an interventional device that is defined by the ROI is registered with a part of a previously acquired image volume that is defined by the ROI. In a first step, a processor requests that the user enter a shape and a size of a region of interest. Based on the parameters entered, the processor defines the shape and size of the ROI. The size of the ROI was discussed above. The shape of the ROI can vary as well. In accordance with one aspect of the present invention, the shape is a cube. The shape can also be non-cubic and can have non-linear sides.

In the second step illustrated in FIG. 3, a catheter is inserted into a patient. The catheter includes at least one sensor. As shown in FIG. 2, the catheter can include a single sensor located, by way of example only, at the tip of the catheter. As shown in FIG. 1, the catheter can also include multiple sensors. A sensor can optionally be located at the tip of the catheter or they can be spaced along the catheter as shown in FIG. 1.

In the third step illustrated in FIG. 3, an image of a section of the patient's body is obtained. The image can be obtained by standard imaging techniques well known to those of ordinary skill in the art.

In the fourth step illustrated in FIG. 3, a location of the sensor or sensors is determined. If the sensor is an active microcoil, then its location can be determined in accordance with the disclosure in “Accurate Localization of Active Devices during Interventional MR Imaging” by Barbot et al. in Proc. Intl. Soc. Mag. Reson. Med. 19 (2011), at page 1747. If the sensor is a passive sensor, it can be located by known segmentation procedures.

In the fifth step illustrated in FIG. 3, the ROI with a defined or selected shape and size that is related to the location of at least one of the sensors (e.g., a microcoil) is applied to the processing. As explained before, the processor determines the ROI based a pre-defined ROI shape or size or based on the selected ROI shape or size. The processor determines the central point of the ROI based on the determined location of the sensor and the pre-defined or selected ROI shape and size through well known calculations.

In the last step illustrated in FIG. 3, the processor registers the part of the image volume defined by the ROI with a part of a previously acquired image volume.

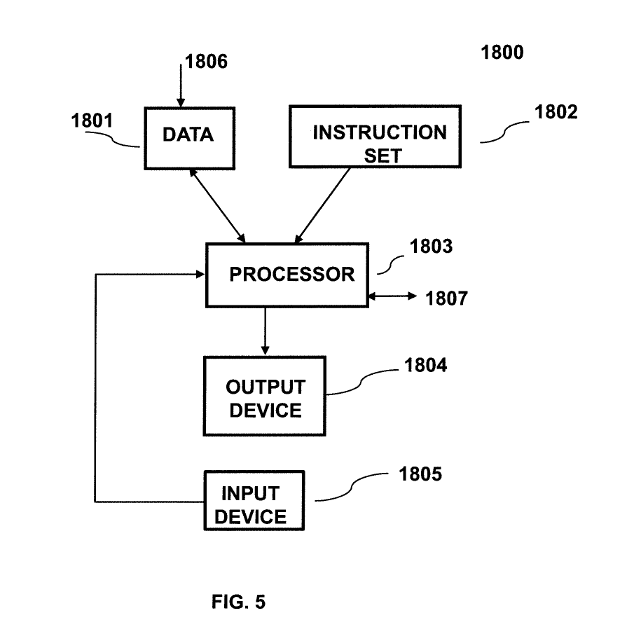

The methods as provided herein are, in one embodiment of the present invention, implemented on a system or a computer device. A system 1800 illustrated in FIG. 5 and as provided herein is enabled for receiving, processing and generating data. The system is provided with data that can be stored on a memory 1801. Data which may be medical image data may be obtained from a medical imaging device such as an MRI machine or data may be provided from a data source. Data may be provided on an input 1806. Such data may be image data, or any other data that is helpful in a context of the present invention. The processor is also provided or programmed with an instruction set or program to execute the methods of the present invention that is stored on a memory 1802 and is provided to the processor 1803, which executes the instructions of 1802 to process the data from 1801 or other input data. Data, such as image data or any other data provided or generated by the processor can be outputted on an output device 1804, which may be a display to display data or images or a data storage device. The output device 1804 in one embodiment is a screen or display where upon the processor displays images such as video images which illustrate contours in a medical image. The processor also has a communication channel 1807 to receive external data from a communication device and to transmit data to an external device. The system in one embodiment of the present invention has an input device 1805, which is an input device such as a keyboard which for instance allows a user to configure the system. An input device which may also be or include a keyboard, a mouse, a pointing device, or the like or any other device that can generate signals representing data to be provided to processor 1803. Thus, the processor illustrated in FIG. 5 can perform all of the steps described above.

The processor can be dedicated hardware. However, the processor can also be a CPU or any other computing device that can execute the instructions of 1802. Accordingly, the system as illustrated in FIG. 5 provides a system for data processing resulting from a medical imaging machine or any other data source and is enabled to execute the steps of the methods as provided herein as an aspect of the present invention.

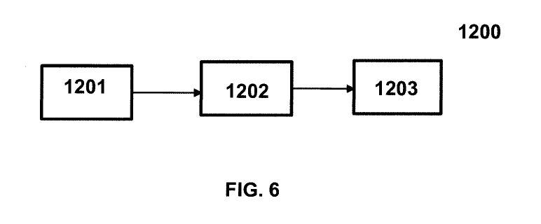

FIG. 6 shows a diagram of a system 1200 which has a data source 1201 which may be an MRI machine or a data source that holds MRI data and which is connected to a computer system 1202. The computer system 1202 which includes a processor is programmed to receive image data from the source 1201 and to process the images in accordance with one or more aspects of the present invention. The processed image data is provided via an output 1205 to a target 1203 which may be a display which shows MRI images or displays related data.

The system 1200 in one embodiment of the present invention is used by a medical organization. The system 1200 in an embodiment of the present invention is located in a medical facility such as a hospital, a clinic, an emergency clinic or a medical practice operated by medical practitioners.

When multiple microcoils are on the catheter, as illustrated in FIG. 1, additional processing steps can be performed in accordance with an aspect of the present invention. Each of the microcoils can have different characteristics so that the individual mircocoils are identifiable. Different ROI's can be established based on the detection of the different sensors in accordance with the steps described above.

In accordance with a further aspect of the present invention, when a microcoil is not provided on the tip of the catheter, the processor determines the location of the tip. In FIG. 4, the type of catheter is entered by the user. When the location of a catheter located a distance from the tip is determined, the processor uses this information to determine the location of the tip of the catheter. The processor knows the distance of the tip of the catheter from the microcoil based on the type of catheter entered in FIG. 4. The processor then uses this information and the location of the microcoil to determine the location of the tip of the catheter.

While there have been shown, described and pointed out fundamental novel features of the invention as applied to preferred embodiments thereof, it will be understood that various omissions and substitutions and changes in the form and details of the methods and systems illustrated and in its operation may be made by those skilled in the art without departing from the spirit of the invention. It is the intention, therefore, to be limited only as indicated by the scope of the claims.

1. A method for determining a region of interest in an image volume of a patient, comprising:
defining on a processor a size and a shape of the region of interest;
applying to the patient a catheter with at least one sensor which is enabled to be detected in the image volume;
the processor receiving data representing the image volume;
the processor determining a location of the at least one sensor in the image volume;
the processor determining the region of interest in the image volume based on the location of the at least one sensor in the image volume;
the processor receiving image data of a previously acquired image volume of the patient; and
the processor registering a part of the previous acquired image volume defined by the region of interest with the region of interest in the image volume.

2. The method of claim 1, wherein the at least one sensor is a microcoil.

3. The method of claim 1, wherein the at least one sensor is a passive sensor.

4. The method of claim 1, wherein the catheter and the at least one sensor is moved relative to the patient to obtain a second location in the image volume and the processor determines a second region of interest.

5. The method of claim 1, wherein the shape of the region of interest depends upon a bounding shape of a location of the catheter in the patient.

6. The method of claim 1, wherein the shape of the region of interest depends upon a geometry of an imaged organ.

7. The method of claim 1, wherein the processor processes image data inside the region of interest.

8. The method of claim 1, wherein the location of the at least one sensor is used for an alignment of the previously acquired image volume with an interventional device.

9. The method of claim 1, wherein the location of the at least one sensor in the image volume is used by the processor as a reference frame relative to the patient.

10. The method of claim 1, wherein the processor determines the region of interest during an interventional procedure on the patient.

11. A system to determine a region of interest in an image volume of a patient, comprising:
a memory enabled to store and retrieve data;
a processor enabled to execute instructions to perform the steps:
defining a size and a shape of the region of interest;
receiving data representing the image volume;
determining a location of at least one sensor of a catheter in the image volume;
determining the region of interest in the image volume based on the location of the at least one sensor in the image volume; and
registering a part of a previously acquired image volume defined by the region of interest with the region of interest in the image volume.

12. The system of claim 11, wherein the at least one sensor is a microcoil.

13. The system of claim 11, wherein the at least one sensor is a passive sensor.

14. The system of claim 11, wherein the at least one sensor is moved relative to the patient to obtain a second location in the image volume and the processor determines a second region of interest.

15. The system of claim 11, wherein the shape of the region of interest depends upon a bounding shape of a location of a catheter in the patient.

16. The system of claim 11, wherein the shape of the region of interest depends upon a geometry of an imaged organ.

17. The system of claim 11, wherein the processor processes image data inside the region of interest.

18. The system of claim 11, wherein the location of the at least one sensor is used for an alignment of the previously acquired image volume with an interventional device.

19. The system of claim 11, wherein the location of the at least one sensor in the image volume is used by the processor as a reference frame relative to the patient.

20. The system of claim 11, wherein the processor determines the region of interest during an interventional procedure on the patient.
